(19) United States
(12) Patent Application Publication
(10) Pub. No.: US 2026/0232608 A1
(43) Pub. Date: Aug. 13, 2026

(54) AMINOTHIOLS FOR THE TREATMENT OF CONDITIONS CHARACTERIZED BY PROTEIN MISFOLDING OR AGGREGATION, MITOCHONDRIAL DYSFUNCTION, OR CHRONIC INFLAMMATION

(71) Applicant: Obvia Pharmaceuticals, Ltd., Madison, WI (US)

(72) Inventors: William FAHL, Madison, WI (US); Torsten GOESCH, Singapore (SG); Hannah Rebecca GOESCH, Gruenwald (DE); Sarah Ricarda GOESCH, Gruenwald (DE)

(21) Appl. No.: 19/154,599

(22) PCT Filed: Feb. 9, 2024
(86) PCT No.: PCT/US2024/015136

§ 371 Date: Aug. 7, 2025

Related U.S. Application Data
(60) Provisional application No. 63/484,320, filed on Feb. 10, 2023.

(51) Int. Cl. A61K 31/145; A61P 21/00; A61P 25/28; A61P 29/00; A61P 37/06; A61P 39/06

(52) U.S. Cl. CPC A61K 31/145; A61P 21/00; A61P 25/28; A61P 29/00; A61P 37/06; A61P 39/06

(57) ABSTRACT

The invention features aminothiols for treating (1) diseases and conditions characterized by misfolding and/or aggregation of endogenous proteins, (2) diseases and conditions associated with mitochondrial dysfunction, and (3) diseases and conditions associated with chronic inflammation in a subject in need thereof.

### FIELD OF THE INVENTION

Provided herein is technology in which the PrC-210 aminothiol, or its close analog, is shown to be very effective when administered to treat (i) diseases and conditions characterized by misfolding and/or aggregation of endogenous proteins, (2) diseases and conditions associated with mitochondrial dysfunction, and (3) diseases and conditions associated with chronic inflammation.

### BACKGROUND

Glutathione (GSH) is the most abundant non-protein thiol in human cells. While synthesized exclusively in the cytosol from its constituent amino acids, GSH is distributed in different compartments, including mitochondria where its concentration in the matrix equals that of the cytosol. It plays a key role in the detoxification of lipid hydroperoxides and electrophiles. Moreover, as mitochondria play a central strategic role in the activation and mode of cell death, mitochondrial GSH has been shown to critically regulate the level of sensitization to secondary insults that induce mitochondrial membrane permeabilization and release of proteins confined in the intermembrane space that once in the cytosol engage the molecular machinery of cell death (see Vicent Ribas et al, Glutathione and mitochondria, Front. Pharmacol., 1 Jul. 2014 Sec. Experimental Pharmacology and Drug Discovery Volume 5-2014). Among its functions, GSH forms transient disulfide bonds with cysteines located on many proteins, and thus can suppress protein misfolding and aggregation. Cysteines are predominantly found in functionally and structurally crucial regions of proteins and play a central role in protein-folding, or when in excess, in protein aggregation. GSH binding (“occupancy”) of protein cysteine residues can protect the cysteine residues, suppress non-natural disulfide bond formation, and thus protect the surrounding protein structure from damage and loss of function. Importantly, the GSH-cysteine binding is reversible (see, e.g., Koji Aoyama, Int. J. Mol. Sci. 2021, 22 (9), 5010). It has been shown that proteins form intra-molecular, non-native, disulfide bonds, which lead to misfolded protein formations (see, e.g., Borges et al., 2014 Jul. 20; 21 (3): 511-531). Although GSH confers this cysteine residue protection, the GSH pool is limited per cell, and GSH doesn't cross the blood-brain barrier; therefore, once the GSH pool is exhausted, it cannot be replaced when the redox balance has been lost in vivo. Because i) GSH pools in brain cells and other body organs are limited, and ii) they are known to be exhausted under oxidative stress conditions, alternative interventions are needed that provide the requisite stability, biodistribution, and redox performance needed to protect cellular structures and inhibit protein misfolding and aggregation. There is a need for therapeutic interventions superior to GSH that i) protect cellular structures, such as proteins, lipids, sugars, DNA and RNA from reactive molecules, ii) inhibit protein misfolding and aggregation and iii) enter and access all cells within a body, and for new methods for treating (i) diseases and conditions characterized by misfolding and/or aggregation of endogenous proteins, (2) diseases and conditions associated with mitochondrial dysfunction, and (3) diseases and conditions associated with chronic inflammation.

### SUMMARY

The invention features PrC-210 aminothiol, or its close analog, for the treatment of (i) diseases and conditions characterized by misfolding and/or aggregation of endogenous proteins, (2) diseases and conditions associated with mitochondrial dysfunction, and (3) diseases and conditions associated with chronic inflammation.

In a first aspect, the invention features a method of inhibiting aggregation of a protein in a subject in need thereof, the method including administering to the subject an effective amount of a compound of formula (I),

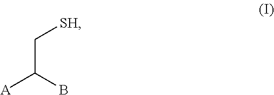

or a pharmaceutically acceptable acid addition salt thereof, in an amount sufficient to inhibit the aggregation of the protein or peptide, and wherein (i) A is —CH₂NHR′ and B is —CH₂NHR, or A=—NRR′ and B═H; and (ii) each of R and R′ are, independently, selected from H, C₁-C₆ alkyl, and C₁-C₆ heteroalkyl, with the proviso that R and R′ are not both H if B═H. In particular embodiments, the compound is

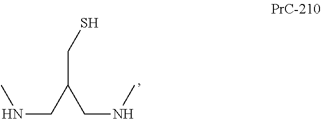

or a pharmaceutically acceptable salt thereof. In some embodiments, the protein for which aggregation is inhibited is selected from TDP-43, alpha-synuclein, tau, amyloid beta, PolyQ htt, islet amyloid polypeptide and prions, or their unaggregated precursors. For example, the subject can have a neurodegenerative disease characterized by misfolding and/or aggregation of endogenous proteins. Neurodegenerative diseases that can be treated using the methods of the invention include, without limitation, Parkinson's disease, prion disease, Alzheimer's disease, multiple system atrophy, Diffuse Lewy body disease, frontotemporal dementia, amyotrophic lateral sclerosis, Huntington's disease, spinocerebellar ataxias and other Poly-Q diseases, hereditary cerebral amyloid angiopathy, and any other neurodegenerative disease described herein. In certain embodiments, the neurodegenerative disease is a prion disease selected from Creutzfeldt-Jakob disease, variant Creutzfeldt-Jakob disease, genetic human prion disease, Bovine Spongiform Encephalopathy (BSE) and Scrapie. In some embodiments, the neurodegenerative disease is a synucleinopathy (e.g., Parkinson's disease (PD), dementia with Lewy bodies (DLB) and multiple system atrophy (MSA)). In particular embodiments, the subject has a disease or condition characterized by amyloidosis. The amyloidosis can be selected from primary systemic amyloidosis (AL amyloidosis), reactive systemic amyloidosis (AA amyloidosis), type II diabetes, injection-localized amyloidosis, beta-2 microglobulin amyloidosis, hereditary non-neuropathic amyloidosis and Finnish hereditary systemic amyloidosis. In other embodiments, the subject has a disease or condition characterized by ocular protein aggregation. For example, the disease or condition characterized by ocular protein aggregation can be cataracts or presbyopia.

In another aspect, the invention features a method of treating a condition associated with mitochondrial dysfunction in a subject in need thereof, the method including administering to the subject an effective amount of a compound of formula (I),

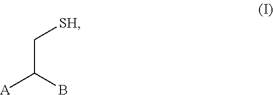

or a pharmaceutically acceptable acid addition salt thereof, in an amount sufficient to inhibit the aggregation of the protein or peptide, and wherein (i) A is —CH₂NHR′ and B is —CH₂NHR, or A=—NRR′ and B═H; and (ii) each of R and R′ are, independently, selected from H, C₁-C₆ alkyl, and C₁-C₆ heteroalkyl, with the proviso that R and R′ are not both H if B═H. In particular embodiments, the compound is

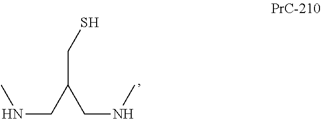

or a pharmaceutically acceptable salt thereof. In some embodiments, the condition associated with mitochondrial dysfunction is a neurodegenerative disorder, a neuropsychiatric disorder, diabetes, metabolic disease, an ocular disorder associated with mitochondrial dysfunction, an ischemia related condition, aging, mitochondrial toxicity associated with therapeutic agents, or migraine. For example, the condition associated with mitochondrial dysfunction can be a neurodegenerative disorder selected from Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, Parkinson's disease, Alzheimer's disease, and Huntington's disease. In some embodiments, the condition associated with mitochondrial dysfunction is a neuropsychiatric disorder selected from bipolar disorder, schizophrenia, depression, addiction disorders, anxiety disorders, attention deficit disorders, personality disorders, autism (i.e., Autism Spectrum Disorder), and Asperger's Syndrome. In some embodiments, the method ameliorates mitochondrial toxicity of therapeutic agents administered to the subject. In certain embodiments, the condition associated with mitochondrial dysfunction is an ocular disorder associated with mitochondrial dysfunction selected from glaucoma, diabetic retinopathy and age-related macular degeneration. In particular embodiments, the condition associated with mitochondrial dysfunction is an ischemia related condition resulting from vascular occlusion, arteriosclerosis, heart valve diseases, tachycardia, or hypo- or hypertension.

In another aspect, the invention features a method of treating a chronic inflammatory condition in a subject in need thereof, the method including administering to the subject an effective amount of a compound of formula (I),

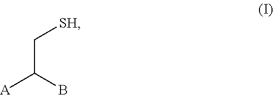

or a pharmaceutically acceptable acid addition salt thereof, in an amount sufficient to inhibit the aggregation of the protein or peptide, and wherein (i) A is —CH₂NHR′ and B is —CH₂NHR, or A=—NRR′ and B═H; and (ii) each of R and R′ are, independently, selected from H, C₁-C₆ alkyl, and C₁-C₆ heteroalkyl, with the proviso that R and R′ are not both H if B═H. In particular embodiments, the compound is

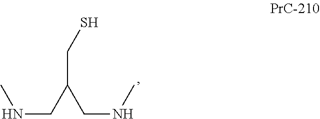

or a pharmaceutically acceptable salt thereof. In some embodiments, the chronic inflammatory condition is selected from rhinosinusitis, arthritis, dermatitis, vasculitis, acute malaria, sickle cell disease, a gastrointestinal inflammatory condition, or an inflammatory pulmonary condition. In particular embodiments, the chronic inflammatory condition is an inflammatory pulmonary condition selected from asthma, chronic obstructive pulmonary disease (COPD), physical trauma-induced pulmonary conditions, emphysema, bronchitis, sarcoidosis, histiocytosis, lymphangiomyomatosis, acute lung injury, chronic lung disease, bronchopulmonary dysplasia, pneumonia, airway exacerbations, and acute respiratory distress syndrome (ARDS). In some embodiments, the chronic inflammatory condition is a gastrointestinal inflammatory condition selected from inflammatory bowel disease (IBD), ulcerative colitis (UC), Crohn's disease (CD), and colitis. In some embodiments, the chronic inflammatory condition is an autoimmune disease. For example, the methods of the invention can be used to treat an autoimmune disease selected from multiple sclerosis, diabetes Type I, rheumatoid arthritis, juvenile rheumatoid arthritis, psoriatic arthritis, pancreatitis, psoriasis, plaque psoriasis, guttate psoriasis, inverse psoriasis, pustular psoriasis, erythrodermic psoriasis, paraneoplastic autoimmune diseases, autoimmune hepatitis, bullous pemphigoid, myasthenia gravis, inflammatory bowel disease, Crohn's disease, ulcerative colitis, celiac disease, thyroiditis, Sjogren's syndrome, Guillain-Barre disease, Raynaud's phenomenon, Addison's disease, primary biliary cirrhosis, primary sclerosing cholangitis, non-alcoholic fatty liver disease, and non-alcoholic steatohepatitis, and diabetes. In some embodiments, the chronic inflammation is in connection with lipid depositions, such as in the case of arteriosclerosis, heart valves sclerosis or leakage or endothelic damage of arteries, vascular damage during dialysis and metabolic syndrome. In particular embodiments the chronic inflammatory condition is a neuroinflammatory disease. Neuroinflammatory diseases that can be treated using the methods of the invention include multiple sclerosis, neuromyelitis optica, anti-myelin oligodendrocyte glycoprotein antibody disorder, autoimmune encephalitis, transverse myelitis, optic neuritis and neurosarcoidosis, but also include all neurodegenerative disease, such as but not limited to alzheimer's disease, parkinson's disease, amyotrophic lateral sclerosis and Huntington's disease.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid.

As used herein, the term “effective amount” refers to an amount sufficient to effect beneficial or desired results, such as clinical results, and, as such, a “therapeutically effective amount” depends upon the context in which it is being applied. For example, in the context of administering a compound disclosed herein to inhibit protein misfolding, protein aggregation, or peptide aggregation, an effective amount of a compound is, for example, an amount sufficient to ameliorate the symptoms or progression of diseases characterized by such misfolding or aggregation. In the context of treating a condition characterized by mitochondrial dysfunction an effective amount of a compound is, for example, an amount sufficient to ameliorate the symptoms or progression of diseases characterized by such mitochondrial dysfunction. In the context of treating a condition characterized by chronic inflammation an effective amount of a compound is, for example, an amount sufficient to ameliorate the symptoms or progression of diseases characterized by such chronic inflammation. The methods of the invention can include systemic (e.g., intravenous) or local administration (e.g., topical or local injection), as needed depending upon the nature of the condition being treated.

As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms or conditions; diminishment of extent of disease, disorder, or condition; stabilizing (i.e., not worsening) state of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “subject,” as used herein, can be a human, non-human primate, or other mammal, such as but not limited to dog, cat, horse, cow, pig, goat, monkey, rat, mouse, and sheep. In preferred embodiments, the subject is a human.

As used herein, the term “pharmaceutical composition” refers to an active compound, formulated together with one or more pharmaceutically acceptable excipients. In some embodiments, a compound of the invention is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, or capsules; and parenteral administration, for example, by subcutaneous, intramuscular, or intravenous injection.

The term “pharmaceutically acceptable excipient,” as used herein, refers to any inactive ingredient (for example, a vehicle capable of suspending or dissolving the active compound) that is biocompatible and suitable for administration to a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-6, 1-4, or 1-2 carbon atoms (e.g., C₁-C₆, C₁-C₄, or C₁-C₂). Examples include, but are not limited to, methyl, ethyl, isobutyl, sec-butyl, tert-butyl, 2-methylpropyl, and 2,2-dimethylpropyl.

The term “C₁-C₆ heteroalkyl,” as used herein, refers to a branched or straight-chain monovalent saturated aliphatic radical containing C, H, and from one or three N atoms.

Other features and advantages of the invention will be apparent from the following Detailed Description, the drawings, and the claims.

### BRIEF DESCRIPTION OF THE DRAWINGS

FIGS. 1(A) and 1(B) show ¹H NMR spectra of PrC-210 after 24 hr in deuterated water at pH 3.0 (FIG. 1a) or pH 9.1 (FIG. 1b). These spectra show that PrC-210 doesn't change its charged structure and remains stable in this pH range for at least 24 hours.

FIG. 2 shows ¹H NMR spectra of PrC-210 in deuterated water at pH 9.0 at 1 hr, 24 hrs, and 72 hrs. These spectra show that PrC-210 exhibits some instability at high pH after 72 hours.

FIG. 3 is a graph illustrating that the pKas of the two amines of PrC-210 are well above (i.e., 3 pH units) a physiological pH (7.2).

FIG. 4 shows two ¹H NMR spectra of PrC-210 in deuterated water containing ammonium acetate at pH 7.0 at 1 hour and at 24 hours. This figure shows that PrC-210 starts to undergo substantial structural changes within hours, which most likely shows the formation of disulfide bonds between two PrC-210 molecules in the presence of the acetate anion.

FIG. 5 shows a ¹H NMR spectra of PrC-210 in deuterated water containing ammonium acetate and the thiol blocker TCEP at pH 7.0 at 1 hour and at 24 hours. This figure shows that PrC-210 in the presence of TCEP remains stable over 24 hours in the presence of acetate anion.

FIG. 6(A) is a graph depicting a PrC-210 half-life of 3.5 hr in acetate buffer at pH 7.2; the PrC-210 half-life in water was previously observed to be days.

FIG. 6(B) shows a reaction scheme depicting the reaction between anionic ammonium acetate and PrC-210 thiol inducing PrC-210-PrC-210 disulfide bond formation. Briefly, (1) the free electron pair of the negatively charged acetate attacks the proton of the thiol on PrC-210, (2) the deprotonated, activated, thiolate anion on the PrC-210 molecule can then (3) react with PrC-210 thiol molecules to form a PrC-210 dimer.

FIG. 6(C) is an image depicting disulfide formation reaction of FIG. 6(B) in the presence of a protein bearing one or more cysteine residues, such as TDP-43.

FIG. 7 is a table depicting the percentage of [14] C-labeled PrC-210 recovered following intravenous administration to male rats. Only minimal metabolism of PrC-210 (1.07%) after 48 hr was observed.

FIG. 8 is an image showing that the consumption of PrC-210 thiol by added 0.5 mM hydrogen peroxide is significantly enhanced (“accelerated”) in a dose-dependent manner by addition of ammonium acetate seconds before hydrogen peroxide.

FIG. 9(A) shows 8-oxo-2′-deoxyguanosine (8-oxo-dG), (B) p53, (C) Cytochrome C, (D) Caspase 8 and (E) Caspase 3/7 marker levels in mouse brain after irradiation with 8.68 Gy. (F) shows Caspase 3/7 in mouse plasma after irradiation with 8.68 Gy. Caspases are elevated with no PrC-210 treatment and are suppressed to background by PrC-210 with 0.5 MTD PrC-210 given 30 min before irradiation with 8.68 Gy.

FIG. 10 shows body weights over time of three SOD1^{G93A} mice. Weight loss was progressive along with onset of ALS motor symptoms and onset of hind-limb paralysis. Administration of systemic PrC-210 (0.1 MTD, IP) was associated with an immediate plateau in body weight loss.

FIG. 11(A) shows Cytochrome C and (B) Caspase 3/7 marker levels in mouse heart after irradiation with 8.68 Gy. PrC-210, (0.3 MTD) was given 24 hours after irradiation with 8.68 Gy.

FIG. 12(A) shows Caspase 1, (B) Complement 3 and (C) Caspase 3/7 marker levels in mouse brain after irradiation with 8.68 Gy. PrC-210 (0.3 MTD) was given 24 hours after irradiation with 8.68 Gy.

FIG. 13(A) shows paralysis score in the EAE mice model, where different twice weekly. intraperitoneal PrC-210 doses were started 24 hours before immunization with MOG peptide, and (B) where different twice weekly intraperitoneal PrC-210 doses were started when first paralysis symptoms were observed.

FIG. 14 shows paw swelling scores in DBA/1J mice where i) twice weekly intraperitoneal PrC-210 doses were started with first paw symptoms, and ii) where topical application of 370 mM PrC-210 was started at a paw swelling score of higher than 10.

### DETAILED DESCRIPTION

This invention features the use of aminothiols for protecting cellular structures against damage and for inhibiting protein misfolding and aggregation in a subject in need thereof. Applicant has discovered that the aminothiols of the invention have in the presence of negative charges an accelerated ability to form disulfide bonds with cysteine residues of endogenous proteins, suppressing the formation of intra-protein Cys-Cys disulfide bonds and their associated protein misfolding and aggregation, in a host of cellular proteins.

### Aminothiols

The aminothiols useful in the methods of the invention can be synthesized, e.g., as described in U.S. Pat. No. 7,314,959.

### Dosing

The administered dose of the compound of the disclosure depends on factors including the route of administration, the disease to be treated, and physical characteristics, e.g., age, weight, and general health, of the subject. Typically, the amount of a compound disclosed herein (e.g., PrC-210) contained within a single dose or multiple doses over a longer period of time may be an amount that effectively treats the disease without inducing significant toxicity. The dosage may be adapted by the clinician in accordance with conventional factors such as the extent of the disease and different parameters of the subject. Typically, a pharmaceutical composition of the disclosure can be administered in an amount from about 0.001 mg up to about 500 mg/kg/day of an aminothiol, such as PrC-210.

### Methods of Treatment

This invention involves systemic administration of the PrC-210 aminothiol, or a close analog, to protect proteins, lipids, sugars, RNA and DNA, as well as to protect cysteines located within proteins and thus prevent protein misfolding and aggregation, and by so-doing, prevent or significantly suppress, a variety of disease states. We have shown in this invention, that the two amines of the aminothiol PrC-210 are both positively charged within a very broad physiological pH range, which makes them highly attracted via electrostatic interaction to negative charges on proteins, lipids, sugars, RNA and DNA. DNA, RNA, lipids, sugars, proteins, lipo- and gluco-proteins, all of which have a number of negative charges on their surface. The two positive charges on PrC-210 will be drawn by complementing charges to these negative charges on cell biomolecules. Whereas the PrC-210 thiol group shows very low reactivity in water in the absence of negative charges and is highly stable as a monomeric aminothiol, we have shown in this invention, that PrC-210 electrostatic interaction with negative charges in close proximity to the aminothiol, significantly changes the stability of the aminothiol and increases (‘activates’) the PrC-210 thiol group. This “activated thiol” then far more readily reacts with other thiols or reactive structures. In addition, the activated aminothiols of this invention then form disulfide bonds with cysteine residues of endogenous proteins, and this teaches that like reaction of PrC-210 with other cell proteins will suppress the formation of intra-protein Cys-Cys disulfide bonds, and their associated protein misfolding and aggregation.

This invention features the administration of the PrC-210 aminothiol, or a close analog, to treat (i) diseases and conditions characterized by misfolding and/or aggregation of endogenous proteins, (2) diseases and conditions associated with mitochondrial dysfunction, and (3) diseases and conditions associated with chronic inflammation.

### Neurodegenerative Diseases

The methods of the invention can be used to treat neurodegenerative diseases characterized by misfolding and/or aggregation of endogenous proteins, such as Parkinson's disease, prion disease, Alzheimer's disease, multiple system atrophy, Diffuse Lewy body disease, frontotemporal dementia, amyotrophic lateral sclerosis, Huntington's disease, spinocerebellar ataxias and other Poly-Q diseases, and hereditary cerebral amyloid angiopathy.

In some embodiments, the prion disease to be treated is selected from Creutzfeldt-Jakob disease, variant Creutzfeldt-Jakob disease, genetic human prion disease, Bovine Spongiform Encephalopathy (BSE) and Scrapie.

In particular embodiments, the neurodegenerative disease to be treated is a synucleinopathy. Synucleinopathies are characterized by intracellular accumulation of protein aggregates, oligomers, protofibrils and fibrils, containing mainly α-synuclein. In the cases of synucleinopathies it is believed that the pathological effects on nerve cells are induced by the formation of oligomeric aggregates of α-synuclein and the subsequent formation of membrane pores. Examples of synucleinopathies include Parkinson's disease (PD), dementia with Lewy bodies (DLB) and multiple system atrophy (MSA).

The methods of the invention can be used to treat conditions involving amyloidosis. Amyloidosis is a general term that describes a number of diseases characterized by the existence of pathological forms of amyloid proteins, often involving extracellular deposition of protein fibrils, which form numerous “amyloid deposits” or “amyloid plaques,” which may occur in local sites or systematically.

In some embodiments, the amyloidosis to be treated is selected from primary systemic amyloidosis (AL amyloidosis), reactive systemic amyloidosis (AA amyloidosis), type II diabetes, injection-localized amyloidosis, beta-2 microglobulin amyloidosis, hereditary non-neuropathic amyloidosis and finnish hereditary systemic amyloidosis.

### Ocular Protein Aggregation Diseases

The methods of the invention can be used to treat ocular diseases and conditions characterized by misfolding and/or aggregation of endogenous proteins, including cataracts and presbyopia.

Presbyopia is the loss of accommodative ability of the eye resulting in the inability to focus on near objects. Stiffening of eye lens and changes in the elasticity of the lens capsule are common causes of presbyopia. Therefore, a pharmacological agent that could prevent or reverse the hardening of the crystalline lens would provide a promising avenue for a novel non-invasive treatment for presbyopia. At the molecular level, proteins known as crystallins play a major role in the stiffening of the eye lens. The lens crystallins comprise three isoforms, a, b, and g and make up 90% of the eye lens protein content. a crystalline (AC), an ATP-independent chaperone and member of the small heat shock protein (sHsp) family, constitutes 40% of the crystallin protein content. It exists as a hetero-oligomer of two subunits, aA-crystallin (AAC) and aB-crystallin (ABC) and its expression is primarily restricted to the eye lens. It recognizes exposed conformational features in partially unfolded lens proteins and sequesters them from one another, thereby reducing the population of aggregation-prone species that would otherwise lead to various age-related vision impairment. The conversion of soluble AC into HMW aggregates is accompanied by a large increase in lens stiffness.

### Mitochondrial Dysfunction

The methods of the invention can be used to treat diseases and conditions characterized by mitochondrial dysfunction. Conditions associated with mitochondrial dysfunction can result in the progressive mosaic appearance of cells with defective electron transport activity in muscle, with cells almost devoid of cytochrome c oxidase (COX) activity interspersed randomly amidst cells with normal activity, and a higher incidence of COX-negative cells in biopsies (e.g., in older subjects). The organism, during aging, or in a variety of conditions associated with mitochondrial dysfunction, is thus faced with a situation in which irreplaceable post-mitotic cells (e.g., neurons, skeletal and cardiac muscle) must be preserved and their function maintained to a significant degree, in the face of an inexorable progressive decline in mitochondrial respiratory chain function. Neurons with dysfunctional mitochondria become progressively more sensitive to insults like excitotoxic injury. Mitochondrial failure contributes to most degenerative diseases (especially neurodegeneration) that accompany aging. Congenital mitochondrial diseases often involve early-onset neurodegeneration similar in fundamental mechanism to disorders that occur during aging of people born with normal mitochondria. The methods and compositions of the invention allow for the amelioration of dysfunctional mitochondria in these irreplaceable post-mitotic cells, allowing for the rescue or improvement of the mitochondrial function in these cells.

Conditions associated with mitochondrial dysfunction include those in which deficits in mitochondrial respiratory chain activity contribute to the development of pathophysiology of such diseases or disorders in a mammal. This includes (1) congenital genetic deficiencies in the activity of one or more components of the mitochondrial respiratory or electron transport chain; and (2) acquired deficiencies in the levels or activities of one or more components of the mitochondrial respiratory chain, wherein such deficiencies are caused by (a) oxidative damage during aging; (b) elevated intracellular calcium; (c) exposure of affected cells to nitric oxide; (d) hypoxia or ischemia; (e) microtubule-associated deficits in axonal transport of mitochondria, or (f) expression of mitochondrial uncoupling proteins.

Common symptoms of mitochondrial dysfunction include cardiomyopathy, muscle weakness and atrophy, developmental delays (involving motor, language, cognitive or executive function), ataxia, epilepsy, renal tubular acidosis, peripheral neuropathy, optic neuropathy, autonomic neuropathy, neurogenic bowel dysfunction, sensorineural deafness, neurogenic bladder dysfunction, dilating cardiomyopathy, migraine, hepatic failure, lactic acidemia, and diabetes mellitus.

### Neuropsychiatric Disorders

The brain requires ten-fold the energy on average of the rest of the body. Many neuropsychiatric disorders may be associated with abnormalities of energy production or mitochondrial dysfunction, in particular. Neuropsychiatric disorders include, without limitation bipolar disorder (BD), schizophrenia, depression, anxiety disorders, attention deficit disorders, addictive disorders, personality disorders, autism and Asperger's Syndrome. The methods and compositions of the invention can be used for the treatment of neuropsychiatric disorders.

### Neurodegenerative Disorders

The methods and compositions of the invention can be used for the treatment of neurodegenerative disorders. Many progressive neurological diseases result from the execution of neurons by mitochondrial apoptosis. Friedrich's ataxia results from a genetic defect in the frataxin gene, which is involved in mitochondrial iron transport (Babcock et al., Science 276:1709 (1997)); human deafness dystonia results from a defect in a small component of the mitochondrial protein import machinery (Koehler et al., Proc. Natl. Acad. Sci. USA 96:2141 (1999)); one well-characterized cause of amyotrophic lateral sclerosis is deficiency in Cu—Zn superoxide dismutase, which is located in the mitochondrial intermembrane space as well as the cytoplasm (Deng et al., Science 261:1047 (1993)). The discovery that several environmental toxins cause Parkinsonism by inhibiting respiratory complex I and promoting the generation of reactive oxygen species has made this complex a focus for research on the basis of Parkinson's disease (Dawson et al., Science 302:819 (2003)). More recently, the mitochondrial protein encoded by PINK 1 has provided a direct link between mitochondria and Parkinson's disease (Valente et al., Science 304:1158 (2004)). Alzheimer's disease is also linked to mitochondrial toxicity through the mitochondrial protein ABAD, a target of amyloid (Lustbader et al., Science 304:448 (2004)). Huntington's disease has been associated with defects in energy metabolism that appear to be widespread, affecting both the brain and peripheral tissues, and arising from mitochondrial dysfunction (Leegwater-Kim et al., NeuroRx 1:128 (2004)). A basic abnormality involved in the pathogenesis of bipolar disorder (BD) is believed to involve energy production and in particular, mitochondrial activity. Evidence from many sources, including, postmortem, genetic, brain imaging and peripheral cell studies support energy deficits and mitochondrial dysfunction as one important causative factor in the development of BD (see Hough et al., Bipolar Disord. 2:145 (2000), Fattal et al., Psychosomatics 47:1 (2006), and Kato et al., Bipolar Disord. 2:180 (2000)).

### Diabetes and Metabolic Disease

The methods and compositions of the invention can be used for the treatment of diabetes and metabolic disease. The central role of mitochondria in metabolism of carbohydrates and fatty acids gives this organelle an important function in diabetes (Maechler et al., Nature 414:807 (2001)). A mouse knockout of an abundant mitochondrial transcription factor has provided a model for 13-cell ablation in juvenile diabetes (Silva et al., Nat. Genet. 26:335 (2000)). Mutations in mtDNA and in PPARγ, a master regulator of mitochondrial biogenesis, are correlated with type II diabetes. Insulin release depends on mitochondrial function as influenced by the expression of the membrane transporter UCP2 (Petersen et al., Science 300:1140 (2003); Zhang et al., Cell 105:745 (2001)). The activity of thiazolidinediones as antidiabetic agents appears to depend on their ability to serve as ligands for PPARγ and its co-activator, PGC-1, in their control of expression of nuclear genes for mitochondrial gene products (Mootha et al., Nature Genet 34:267 (2003); Puigserver et al., Endocr. Rev. 24:78 (2003)).

### Migraine

The methods and compositions of the invention can be used for the treatment of migraine. Metabolic studies on patients with recurrent migraine headaches indicate that deficits in mitochondrial activity are commonly associated with this disorder, manifesting as impaired-oxidative phosphorylation and excess lactate production. Such deficits are not necessarily due to genetic defects in mitochondrial DNA. Migrainers are hypersensitive to nitric oxide, an endogenous inhibitor of cytochrome c oxidase. In addition, patients with mitochondrial cytopathies, e.g., MELAS, often have recurrent migraines.

Ocular Disorders Associated with Mitochondrial Dysfunction

The methods and compositions of the invention can be used for the treatment of ocular disorders, such as glaucoma, diabetic retinopathy and age-related macular degeneration. Retinal damage is attributed to free radical initiated reactions in glaucoma, diabetic retinopathy and age-related macular degeneration (AMD). The eye is a part of the central nervous system and has limited regenerative capability. The retina is composed of numerous nerve cells which contain the highest concentration of polyunsaturated fatty acids (PFA) and subject to oxidation. Free radicals are generated by UV light entering the eye and mitochondria in the rods and cones, which generate the energy necessary to transform light into visual impulses. Free radicals cause peroxidation of the PFA by hydroxyl or superoxide radicals which in turn propagate additional free radicals. The free radicals cause temporary or permanent damage to retinal tissue.

Glaucoma is usually viewed as a disorder that causes an elevated intraocular pressure (IOP) that results in permanent damage to the retinal nerve fibers, but a sixth of all glaucoma cases do not develop an elevated IOP. This disorder is now perceived as one of reduced vascular perfusion and an increase in neurotoxic factors. Recent studies have implicated elevated levels of glutamate, nitric oxide and peroxynitirite in the eye as the causes of the death of retinal ganglion cells.

Diabetic retinopathy occurs when the underlying blood vessels develop microvascular abnormalities consisting primarily of microaneurysms and intraretinal hemorrhages. Oxidative metabolites are directly involved with the pathogenesis of diabetic retinopathy and free radicals augment the generation of growth factors that lead to enhanced proliferative activity. Nitric oxide produced by endothelial cells of the vessels may also cause smooth muscle cells to relax and result in vasodilation of segments of the vessel. Ischemia and hypoxia of the retina occur after thickening of the arterial basement membrane, endothelial proliferation and loss of pericytes. The inadequate oxygenation causes capillary obliteration or nonperfusion, arteriolar-venular shunts, sluggish blood flow and an impaired ability of RBCs to release oxygen. Lipid peroxidation of the retinal tissues also occurs as a result of free radical damage.

### Ischemia Related Conditions

The methods and compositions of the invention can be used for the treatment of ischemia related conditions. Oxygen deficiency results in both direct inhibition of mitochondrial respiratory chain activity by depriving cells of a terminal electron acceptor for cytochrome c reoxidation at Complex IV, and indirectly, especially in the nervous system, via secondary post-anoxic excitotoxicity and nitric oxide formation. In conditions like cerebral anoxia, angina crises, tissues are relatively hypoxic. In such cases, an increase in mitochondrial activity provides protection of affected tissues from deleterious effects of hypoxia, attenuate secondary delayed cell death, and accelerate recovery from hypoxic tissue stress and injury. The methods and compositions of the invention can be useful for preventing delayed cell death (apoptosis in regions like the hippocampus or cortex occurring about 2 to 5 days after an episode of cerebral ischemia) after ischemic or hypoxic insult, for example, to the brain.

### Muscle Function

The methods and compositions of the invention can be used for enhancing muscle performance. For example, the methods and compositions of the invention may be useful for improving physical endurance (e.g., ability to perform a physical task such as exercise, physical labor, sports activities, etc.), inhibiting or retarding physical fatigues, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood.

Enhanced sports performance, strength, speed and endurance are typically measured by an increase in muscular contraction strength, increase in amplitude of muscle contraction, shortening of muscle reaction time between stimulation and contraction, the ability to overcome muscle fatigue, and ability to maintain activity for longer periods of time. Aside from muscle performance during endurance exercise, free radicals and oxidative stress parameters are affected in pathophysiological states. A substantial body of data now suggests that oxidative stress contributes to muscle wasting or atrophy in pathophysiological states (see Clarkson, Crit. Rev. Food Sci. Nutr. 35:31 (1995); and Powers et al., Proc. Nutr. Soc. 58:1025 (1999)). For example, in muscular dystrophies dystrophin-glycoprotein complex (DGC) defects suggest that one mechanism of cellular injury is functional ischemia related to alterations in cellular NOS and disruption of a normal protective action of NO. Rando (Microsc. Res. Tech. 55:223 (2001)) has shown that oxidative injury precedes pathologic changes and that muscle cells with defects in the DGC have an increased susceptibility to oxidant challenges. Excessive lipid peroxidation due to free radicals has also been shown to be a factor in myopathic diseases such as McArdle's disease (see Russo et al., Med. Hypotheses. 39:147 (1992)). Furthermore, mitochondrial dysfunction is a well-known correlate of age-related muscle wasting (sarcopenia) and free radical damage has been suggested, though poorly investigated, as a contributing factor (see Navarro et al., Front. Biosci. 6: D26 (2001)). Other indications include acute sarcopenia, for example muscle atrophy and/or cachexia associated with burns, bed rest, limb immobilization, or major thoracic, abdominal, and/or orthopedic surgery. The methods of the present invention can be effective in the treatment of muscle related pathological conditions.

### Aging

The methods and compositions of the invention can be used for the treatment of aging and conditions associated therewith. During normal aging, there is a progressive decline in mitochondrial respiratory chain function. Beginning about age 40, there is an exponential rise in accumulation of mitochondrial DNA defects in humans, and a concurrent decline in nuclear-regulated elements of mitochondrial respiratory activity. Many mitochondrial DNA lesions have a selection advantage during mitochondrial turnover, especially in post-mitotic cells. The proposed mechanism is that mitochondria with a defective respiratory chain produce less oxidative damage to themselves than do mitochondria with intact functional respiratory chains (mitochondrial respiration is the primary source of free radicals in the body). Therefore, normally functioning mitochondria accumulate oxidative damage to membrane lipids more rapidly than do defective mitochondria, and are, therefore, “tagged” for degradation by the autophagic and lysosomal systems. The inexorable decline of mitochondrial function with age contributes to the aging-related conditions of neurodegeneration, and type II diabetes. Just as oxidative stress underlies some of these defined diseases, it is thought to contribute to generalized aging (Harman, Proc. Natl. Acad. Sci. USA 78:7124 (1981)). Mutations in C. elegans and D. melanogaster that reduce mitochondrial oxidative stress have been shown to prolong lifespan in these organisms (Hekimi et al., Science 299:1351 (2003)). Moreover, mammals maintained on calorie-restricted diets have a reduced metabolic rate that is thought to contribute to significantly increased longevity. Numerous studies have documented an increase in point mutations and deletions in mtDNA with advancing age. Furthermore, Trifunovic et al. have recently showed that mice engineered to express an error prone mitochondrial DNA polymerase can serve as an excellent model for premature ageing (Nature 429:417 (2004)). The methods of the invention can be used to treat progeria, Werner syndrome, Wiedemann-Rautenstrauch syndrome, and symptoms of aging (e.g., wrinkles, forgetfulness, arthrosis, age-related muscle wasting).

In particular embodiments, the methods of the invention are used to treat a condition characterized by mitochondrial dysfunction selected from a neurodegenerative disorder (e.g., Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, Parkinson's disease, Alzheimer's disease, or Huntington's disease), a neuropsychiatric disorder (e.g., bipolar disorder, schizophrenia, depression, addiction disorders, anxiety disorders, attention deficit disorders, personality disorders, autism, or Asperger's Syndrome), diabetes, metabolic disease, an ocular disorder associated with mitochondrial dysfunction (e.g., glaucoma, diabetic retinopathy or age-related macular degeneration), an ischemia related condition (e.g., a condition resulting from vascular occlusion, arteriosclerosis, heart valve diseases, tachycardia, or hypotension), aging, mitochondrial toxicity associated with therapeutic agents, or migraine.

### Chronic Inflammation

The methods and compositions of the invention can be used for the treatment of chronic inflammation and conditions associated therewith. Exemplary inflammatory conditions that can be treated using the methods of the invention include asthma (e.g., aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, etc.), airway hyperreactivity, airway hyperresponsiveness, rhinosinusitis, rhinosinusitis with polyps, nasal polyposis, arthritis (e.g., osteoarthritis, rheumatoid arthritis, arthritic joints as a result of injury, etc.), seronegative enthesopathy and arthropathy (SEA) syndrome, acute malaria, sickle cell disease, osteoporosis, eosinophilic esophagitis, dermatitis, atopic dermatitis, allergic rhinitis, bullous pemphigoid, chronic urticaria, cartilage inflammation, polymyalgia rheumatic, polyarteritis nodossa, Wegener's granulamatosis, Behcet's disease, myolitis, polymyolitis, dermatomyolitis, dermatomyositis, vasculitis, arteritis, diabetic nephropathy, interstitial cystitis, gastrointestinal inflammatory conditions (e.g., inflammatory bowel disease (IBD), ulcerative colitis (UC), Crohn's disease (CD), colitis (e.g., colitis caused by environmental insults (e.g., caused by or associated with a therapeutic regimen, such as chemotherapy, radiation therapy, etc.), chronic hepatis (e.g. viral hepatitis, alcohol related hepatitis, and chronic hepatis (e.g. viral hepatitis, alcohol related hepatitis, non-alcohol fatty hepatitis, etc.), ischemic colitis, collagenous or lymphocytic colitis, colitis in conditions such as chronic granulomatous disease or celiac disease, food allergies, and gastritis, and inflammatory pulmonary conditions (e.g., chronic obstructive pulmonary disease (COPD), allergen-induced pulmonary conditions, pollutant-induced pulmonary conditions (e.g., asbestosis, silicosis, or berylliosis), gastric aspiration-induced pulmonary conditions, immune dysregulation, inflammatory conditions with genetic predisposition such as cystic fibrosis, physical trauma-induced pulmonary conditions (e.g., ventilator injury), emphysema, bronchitis, sarcoidosis, histiocytosis, lymphangiomyomatosis, acute lung injury, chronic lung disease, bronchopulmonary dysplasia, pneumonia (e.g., community-acquired pneumonia, nosocomial pneumonia, ventilator-associated pneumonia, and severe pneumonia), airway exacerbations, and acute respiratory distress syndrome (ARDS). Other examples of chronic inflammatory conditions are disease with neuroinflammation, such as multiple sclerosis and neurodegenerative disease.

### Autoimmune Disease

The methods and compositions of the invention can be used for the treatment of autoimmune conditions, including multiple sclerosis, diabetes Type I, rheumatoid arthritis, juvenile rheumatoid arthritis, psoriatic arthritis, pancreatitis, psoriasis, plaque psoriasis, guttate psoriasis, inverse psoriasis, pustular psoriasis, erythrodermic psoriasis, paraneoplastic autoimmune diseases, autoimmune hepatitis, bullous pemphigoid, myasthenia gravis, inflammatory bowel disease, Crohn's disease, ulcerative colitis, celiac disease, thyroiditis (e.g., Graves' disease), Sjogren's syndrome, Guillain-Barre disease, Raynaud's phenomenon, Addison's disease, liver diseases (e.g., primary biliary cirrhosis, primary sclerosing cholangitis, non-alcoholic fatty liver disease, and non-alcoholic steatohepatitis), and diabetes (e.g., type I diabetes).

### Related Conditions

The methods and compositions of the invention can be used for the treatment of conditions exacerbated by one or more of protein aggregation, mitochondrial dysfunction, and/or chronic inflammation, including encephalitis, meningitis, nephritis, myocarditis, pneumonia, gangrene and non-healing wounds, chronic hepatitis, endotoxin liver or organ damage, acne, inflammatory bowel diseases, colitis ulcerosa, Morbus Crohn, psoriasis, vasculitis, glomerulonephritis, pancreatitis, tendinopathies, thyroiditis, myasthenia gravis, pernicious anemia, Dermatomyositis, Sjörgren syndrome, aplastic anemia, coeliac disease, eczema, Arteriosclerosis, hypertension, myocardial ischemia, neural ischemia, kidney ischemia, chronic heart failure and cardiac hypertrophy, cardiomyopathies, chronic kidney disease, coronary heart disease, stroke, aortic disease, varicose veins, angina, organ failure (e.g., kidney, liver and/or lung failure), fatty strips formations, atheroma, atherosclerosis plaques, fetal growth restriction, and preeclampsia.

The following examples are meant to illustrate the invention. They are not meant to limit the invention in any way.

### Examples

### Example 1: Mode of Action of the Invention

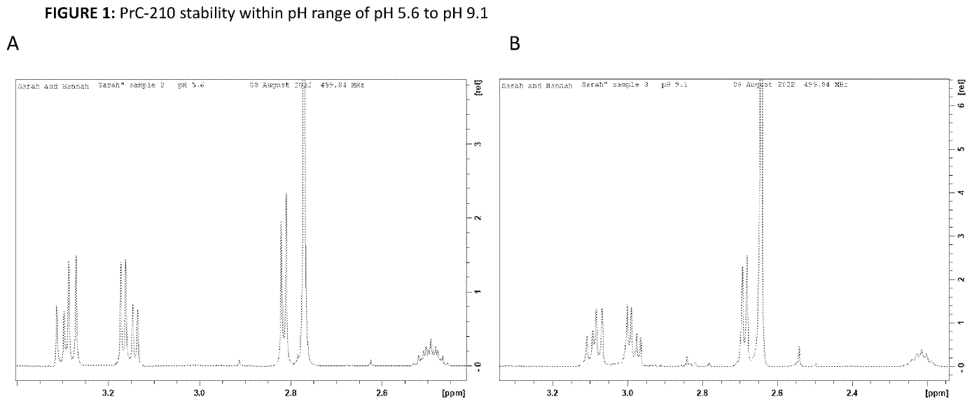

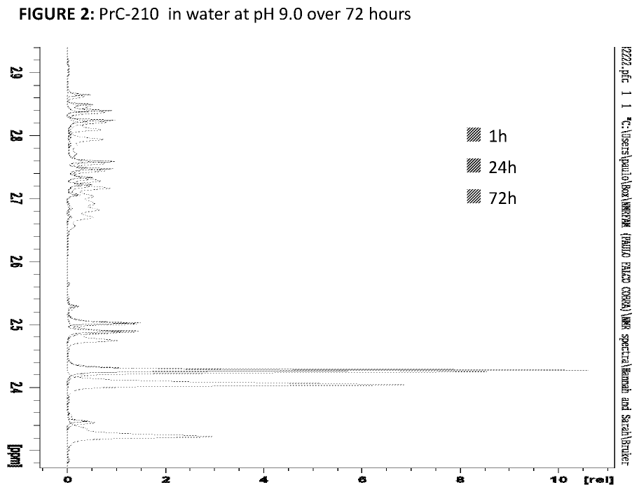

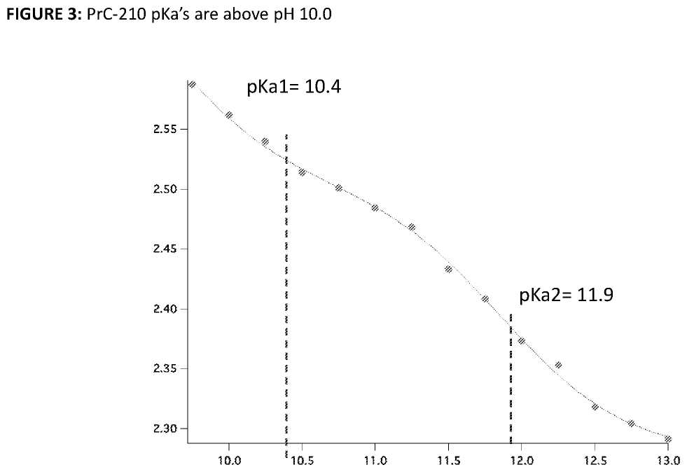

We established the chemical structure profile of PrC-210 using Nuclear Magnetic Resonance (NMR) spectroscopy experiments. We discovered that when the solution pH was titrated in water between pH 3 and pH 9.1 that PrC-210 doesn't change its charged structure and remains stable (see FIGS. 1a and 1b). This structural stability of the PrC-210 mono-thiol form in this pH range remained for at least the observed 24 hours. We did see a significant change after 72 hours at pH 9.0 (FIG. 2). Adding the thiol blocker TCEP in water doesn't influence the PrC-210 stability over the 72 hr at pH 9.0 (data not shown), which means that the thiol group is stable and not reactive (or, ‘activated’) under these conditions. In our pH titrations between pH 9.0 and 13.0, we saw clear structural changes to PrC-210; from these progressive changes, we calculated that the pKas of the two PrC-210 amines are all substantially above the pH (pH 7.2) of physiological conditions (FIG. 3).

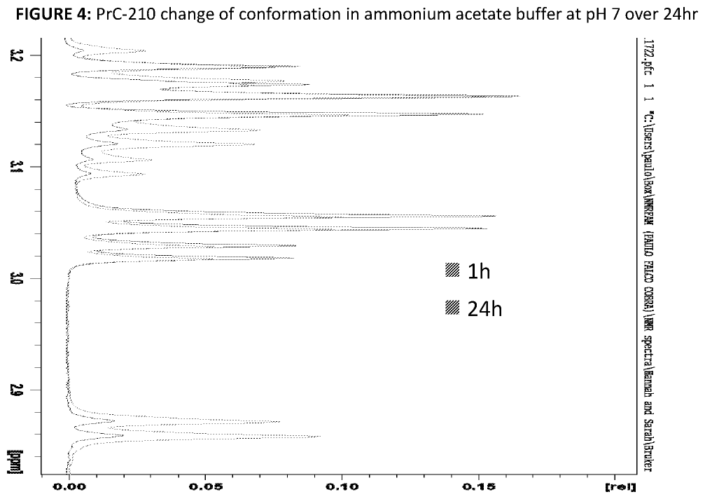

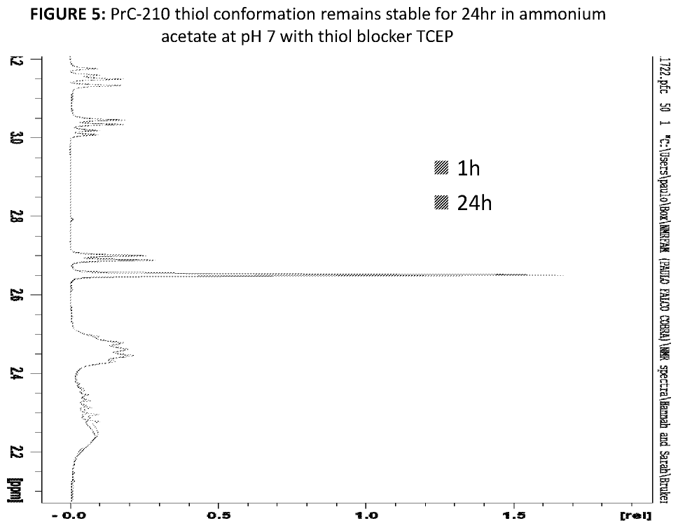

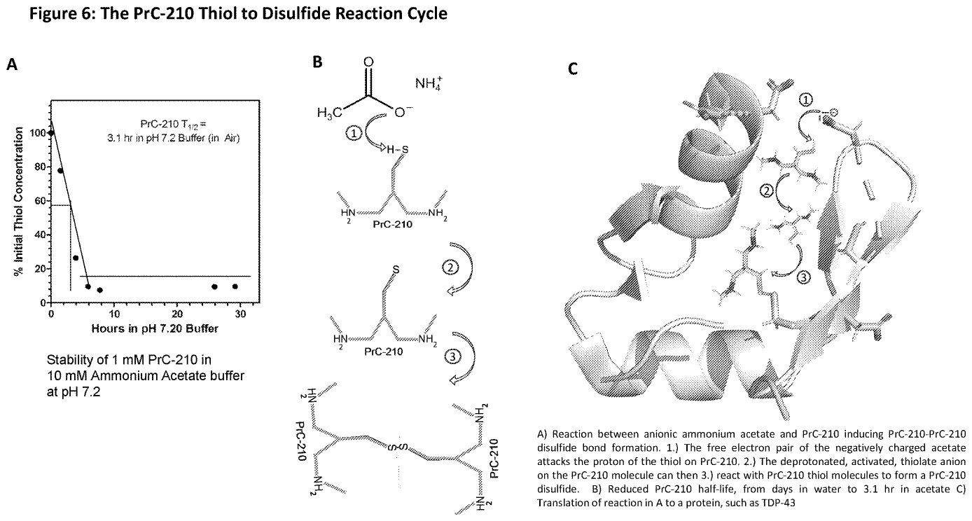

Interestingly, when ammonium acetate, which contains anionic charges, is added at pH 7, PrC-210 starts to undergo substantial structural changes within hours (FIG. 4), which indicates the formation of disulfide bonds between two PrC-210 molecules. We checked this hypothesis by adding the thiol blocker TCEP to the ammonium acetate incubation, and single PrC-210 again remained stable for 24 hours (FIG. 5). In FIG. 6A, when ammonium acetate was added to the aqueous PrC-210 mono-thiol solution, we established a half-life of 3.1 hours at pH 7.2. These observations show that when the anionic ammonium acetate molecule is added to an aqueous PrC-210 mono-thiol solution, thiol reactivity greatly increases, and this yields formation of PrC-210 disulfide molecules; i.e., 1) the free electron pair of the negatively charged acetate attacks the proton of the thiol on PrC-210, and that 2) the deprotonated, activated thiolate anion on the PrC-210 molecule then develops an affinity to 3) react with another PrC-210 thiol to form a PrC-210 disulfide dimer (FIG. 6B).

We expect that the thiol activation in the presence of negatively charged ammonium acetate reflects the reaction which occurs when a single PrC-210 is in close proximity to any negatively charged molecule or site. The thiol is then activated to react with any of several reactive sites, including electrophiles, other thiol residues on PrC-210 molecules, or other thiols on Cys residues of cell proteins to form disulfides (FIG. 6C), carbon structures (thioether), oxygen structures to form Sulfoxide (1xO), Sulfenic ((1xO) H), Sulfinic ((2xO) H) and Sulfonic acid ((3xO) H), nitrogen structures to form S-nitrosothiol or non-radicals such as hydrogen peroxide.

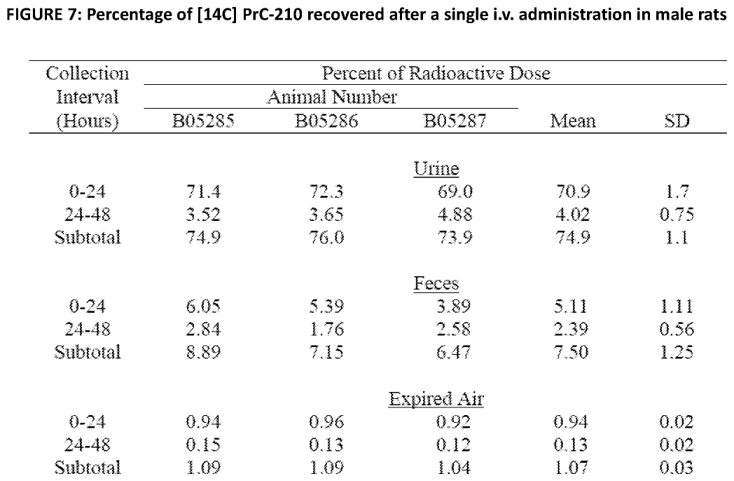

We were also interested to see whether PrC-210-cysteine disulfide formation is transient or permanent; permanent PrC-210 hetero-disulfide formation with cellular proteins would be expected to have an impact on protein function and could translate into unwanted, toxic, side effects and should be seen as PrC-210 residues showing up as urinary or fecal metabolites. Several experimental results indicate no discernible evidence of permanent PrC-210-cysteine/protein disulfide formation, these include: i) there are no toxic side effects from PrC-210 within its therapeutic window (see, e.g., Soref et al., Int J Radiation Oncol Biol Phys, Vol. 82, No. 5, pp. e701ee707, 2012), and ii) in a pharmacokinetics study using [14] C labeled PrC-210, we found only minimal metabolism of PrC-210 (1.07%) after 48 hr (FIG. 7, unpublished data). It is well described that i) disulfide binding is reversible (see, e.g., Koji Aoyama, Int. J. Mol. Sci. 2021, 22 (9), 5010) and ii) that there are also two highly efficient thiol reducing systems in the cell (see, e.g., Cumming et al., The Journal of Biological Chemistry vol. 279, No. 21, Issue of May 21, pp. 21749-21758, 2004), which reduce disulfide bonds between glutathione and proteins in non-oxidative stress situations (see, e.g., Koji Aoyama, Int. J. Mol. Sci. 2021, 22 (9), 5010). Based upon both our experimental findings and the published literature, it would then be logical that PrC-210 disulfide bonds with proteins would be governed by the same chemical and enzymatic processes.

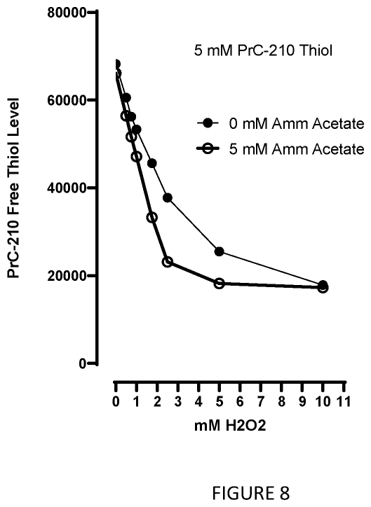

We believe that the increased reactivity of the thiol in the presence of negative charges, such as ammonium acetate, also occurs when PrC-210-thiol is in close proximity to other reactive structures, such as hydrogen peroxide. We have studied hydrogen peroxide, as it is a non-radical oxygen species with a much longer half-life than superoxide. We added hydrogen peroxide to an aqueous 5 mM solution of PrC-210 thiol at pH 7. Hydrogen peroxide caused a dose-dependent consumption of free PrC-210 thiol (FIG. 8). Addition of 5 mM ammonium acetate to the aqueous 5 mM solution of PrC-210 thiol at pH 7, seconds before the hydrogen peroxide, caused a significant enhancement of the hydrogen peroxide consumption of PrC-210 thiol (an “accelerant effect”).

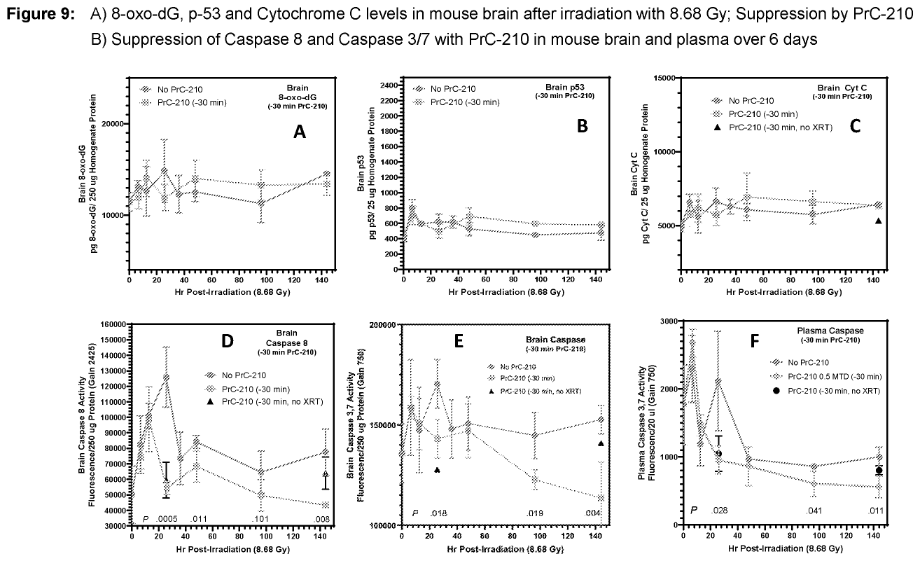

To examine PrC-210-conferred protection in an animal model, we irradiated mice with 8.68 Gy and treated them with a 0.5MTD dose of PrC-210 30 minutes before irradiation. Normally, lethal radiation with 8.68 Gy leads to massive DNA damage and activation of the intrinsic apoptotic pathway. When we examined the brains of the irradiated mice (FIG. 9A-C), neither 8-oxo-dG, Cytochrome C nor p53 damage biomarker levels were significantly elevated. This means that the radiation insult didn't trigger a significant activation of the intrinsic apoptotic pathway nor did it trigger a mitotic arrest with p53 activation. The most likely reason for this is that neuronal cells are already in a most-mitotic stage and therefore the DNA is highly protected against radiation insults. Interestingly, caspase 8 and caspase 3/7 were activated without PrC-210 and were suppressed to background when PrC-210 was administered systemically 24 hours before the radiation insult, which means that proteins were protected against damage with PrC-210 present (FIG. 9D, E). This apoptotic suppression and protection lasted for the entire observation time of six days, which is a sign, that PrC-210 was still present after six days. When we looked at mouse plasma in the same experiment, we saw the same 6 days apoptotic suppression as in the brain in caspase 3/7 levels (FIG. 9F).

In cells, the plus-charged amines on PrC-210 will generally i) cause the PrC-210 molecule to concentrate and “hover” around minus-charged biomolecules, and ii) the biomolecule minus charges will also increase reactivity of the PrC-210 thiol, thus facilitating its reaction with reactive structures, such as hydroperoxide, and other thiol groups on cell proteins. We conclude that for the treatment of diseases: i) the amount of PrC-210 required at a certain moment of time is dependent on the activity of the disease, ii) the amount of PrC-210 required in a steady state might be relatively small and therefore PrC-210 available in a cell works as a reservoir, and iii) PrC-210, once in a cell, will stay there for a long time as a stable molecule with low reactivity until it is activated through the described mechanism. Therefore, PrC-210 dosing regimens need to take into account acute and chronic nature of the disease and the severity of an insult.

### Example 2: Use of the Invention to Diminish Progression of Diseases with Protein Aggregation, Such as Amyotrophic Lateral Sclerosis

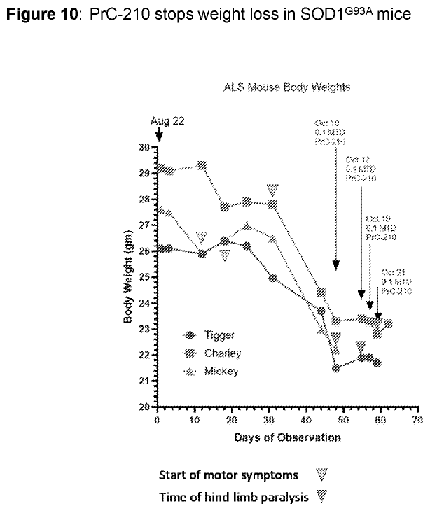

This experiment was designed to illuminate whether PrC-210 is able to stop the progression of diseases which are based on protein aggregation, such as Amyotrophic Lateral Sclerosis (ALS). FIG. 10 shows the body weights over time of SOD1^{G93A} mice. Weight loss was progressive and was in exponential decline along with onset of ALS motor symptoms. The administration of systemic PrC-210 (0.1 MTD, IP) was associated with an immediate plateau in body weight, i.e., weight loss in the ALS mice stopped. As ALS pathology is based upon protein aggregation, the findings in this experiment support our inventive concept that PrC-210 is able to suppress protein misfolding and thereby suppress the pathology of these diseases. This invention applies to all neuronal, organ specific and systemic diseases which are caused by protein aggregations, such as neurodegenerative diseases, local and systemic amyloidosis, Type II diabetes, cataract of the eye, etc.

### Example 3: Use of the Invention to Protect the Mitochondrial Membrane

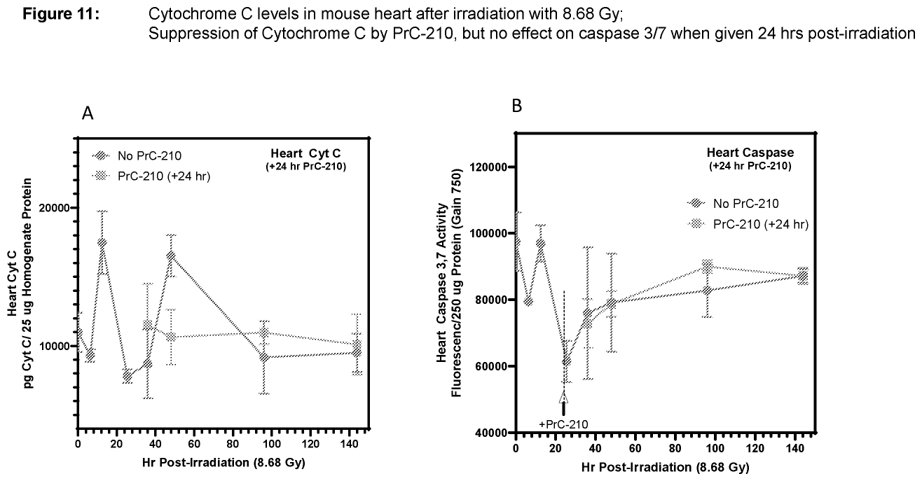

FIG. 11A shows a second cytochrome C peak in mice heart muscle 48 hours post-irradiation with 8.68 Gy. Different from the first peak after 12 hours, this peak is not due to DNA damage and activation of the intrinsic apoptotic pathway, as there is neither an associated p53 nor 8-oxo-dG increase at 48 hours post-irradiation (data not shown), nor an activation of the intrinsic apoptotic pathway (FIG. 11B). This peak represents a massive damage of complex IV of the respiratory chain, which is located at the inner mitochondrial membrane. PrC-210 given 24 hours post-irradiation after the first cytochrome C release is able to penetrate the mitochondria and suppress cytochrome C release to background (FIG. 11A). This discovery teaches us, that PrC-210 enters mitochondria and protects the proteins and lipids of the mitochondrial membranes against insults, insults which result in the release of cytochrome C. This invention applies to all diseases which are caused by damage of the mitochondria, such as aging and all age-related diseases, neurodegenerative diseases, diseases of the cardio-vascular system, such as arteriosclerosis, damage of the heart valves or main arteries, and in general to diseases caused by damage of the mitochondria membranes, the mitochondria DNA and proteins.

### Example 4: Use of the Invention to Protect Brains Directly Against Neuro-Inflammation and to Directly Suppress Chronic Inflammation and Inflammation from Autoimmune Diseases

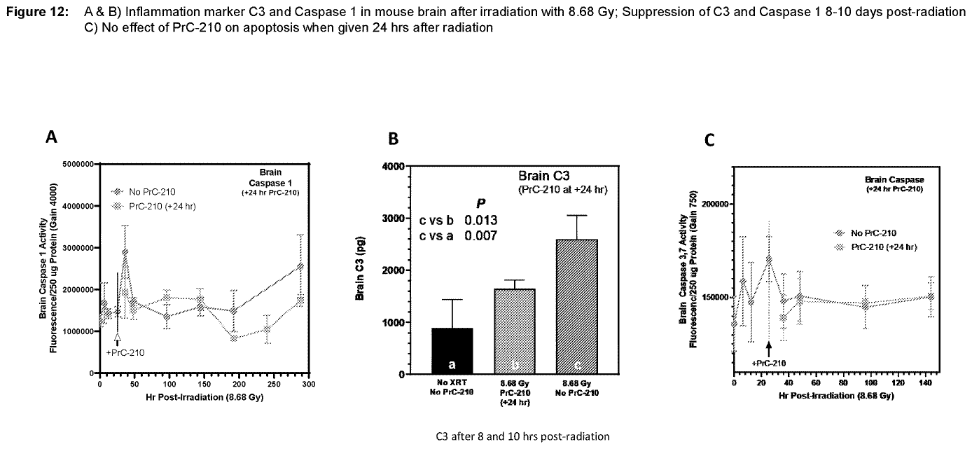

To visualize the effect of radiation on the immune response of the mouse brain, ICR mice received 8.68 Gy whole-body radiation (FIG. 10) at Time “0.” O.3MTD, i.p. PrC-210 was administered 24 hours after irradiation. As shown in FIG. 12A, the inflammasome marker caspase 1 started to be significantly suppressed eight days after radiation. The same applied to the inflammation marker, complement 3 (C3) (FIG. 12B). Treatment with PrC-210 significantly suppressed both inflammation makers. Most important, this suppression was a direct suppression of the immune response and was not an indirect suppression through protection of proteins and lipids against damage. Radiation-induced protein damage would have triggered an apoptotic response, as it can be seen in the second peak of caspase 3/7 in FIG. 12C. Therefore, this experiment teaches that PrC-210 is able to enter the brain and directly suppresses post-irradiation inflammation.

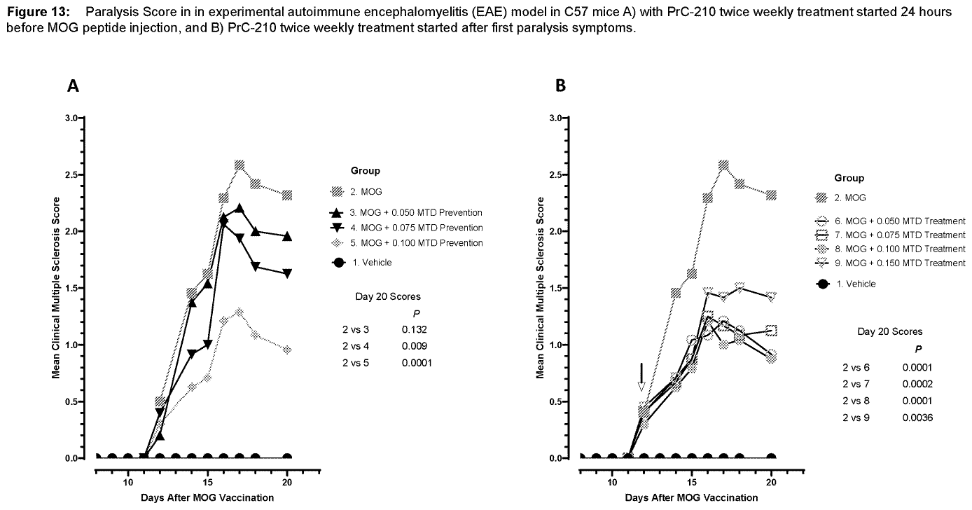

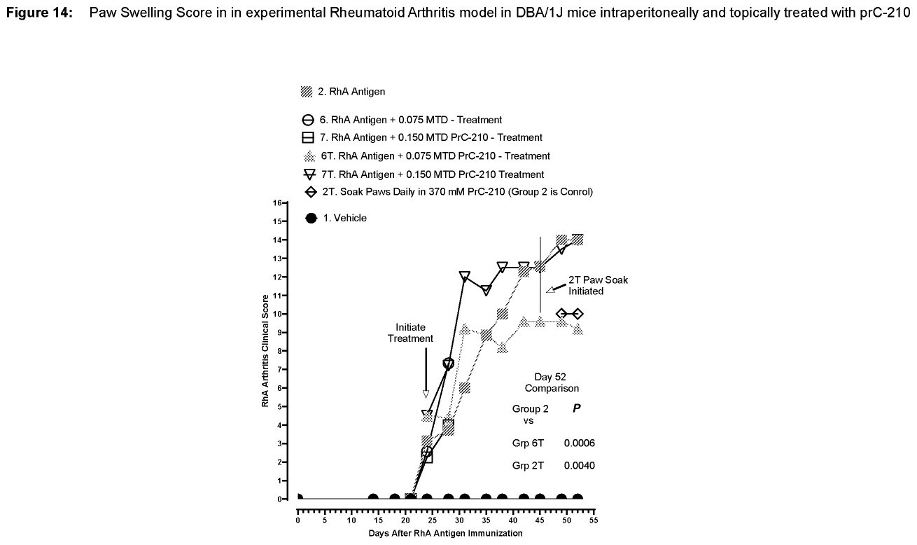

To show, that PrC-210 suppresses neuroinflammation which is caused by an autoimmune induction of the disease, PrC-210 was applied 24 hours before immunization of C57 mice against MOG peptide (FIG. 13A), and immediately after first paralysis symptoms developed from the autoimmunization against myelin. PrC-210 was significantly suppressing the development and the severity of the paralysis symptoms in both application models.

To show further, that this suppression of chronic inflammation applies generally to other organs than the brain, DBA/1J mice immunized against collagen III where treated systemically and topically with PrC-210. The typical swelling of paws induced in this mice model was significantly reduced with twice weekly intraperitoneal injection of 0.075 MTD PrC-210 and 370 mM PrC-210 applied topically.

This invention applies to all diseases which are caused by neuro-inflammation and inflammation, such as i) neuro-degenerative diseases and multiple sclerosis, and the treatment of neuroinflammation when Pr-210 is applied after insults of the brain caused by traumata, irradiation, neurotoxins, ischemia and ischemic-reperfusion injury and microbial diseases, and ii) to all diseases which are caused by inflammation, such as autoimmune diseases, including but not limited to Type I diabetes, rheumatoid arthritis, colitis, psoriasis, lupus erythematodes; pancreatitis, hepatitis, the treatment and prevention of inflammations due to lipid depositions, such as arteriosclerosis, damage of the heart valves or main arteries, vascular damage during dialysis and metabolic syndrome.

### OTHER EMBODIMENTS

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of inhibiting aggregation of a protein in a subject in need thereof, said method comprising administering to the subject an effective amount of a compound of formula (I), 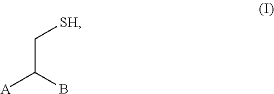
or a pharmaceutically acceptable acid addition salt thereof, in an amount sufficient to inhibit the aggregation of the protein or peptide, and wherein
(i) A is —CH₂NHR′ and B is —CH₂NHR, or A=—NRR′ and B═H; and
(ii) each of R and R′ are, independently, selected from H, C₁-C₆ alkyl, and C₁-C₆ heteroalkyl,
with the proviso that R and R′ are not both H if B═H.

2. The method of claim 1, wherein the compound is 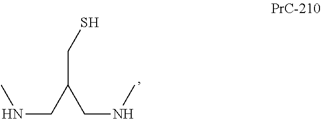
or a pharmaceutically acceptable salt thereof.

3. The method of claim 1 or 2, wherein the protein is selected from TDP-43, alpha-synuclein, tau, amyloid beta, PolyQ htt, islet amyloid polypeptide and prions.

4. The method of claim 1 or 2, wherein the subject has a neurodegenerative disease characterized by misfolding and/or aggregation of endogenous proteins.

5. The method of claim 4, wherein the neurodegenerative disease is Parkinson's disease, prion disease, Alzheimer's disease, multiple system atrophy, Diffuse Lewy body disease, frontotemporal dementia, amyotrophic lateral sclerosis, Huntington's disease, spinocerebellar ataxias and other Poly-Q diseases, or hereditary cerebral amyloid angiopathy.

6. The method of claim 5, wherein the neurodegenerative disease is a prion disease selected from Creutzfeldt-Jakob disease, variant Creutzfeldt-Jakob disease, genetic human prion disease, Bovine Spongiform Encephalopathy (BSE) and Scrapie.

7. The method of claim 4, wherein the neurodegenerative disease is a synucleinopathy.

8. The method of claim 7, wherein the synucleinopathy is selected from Parkinson's disease (PD), dementia with Lewy bodies (DLB) and multiple system atrophy (MSA).

9. The method of claim 1 or 2, wherein the subject has a disease or condition characterized by amyloidosis.

10. The method of claim 9, wherein the amyloidosis is selected from primary systemic amyloidosis (AL amyloidosis), reactive systemic amyloidosis (AA amyloidosis), type II diabetes, injection-localized amyloidosis, beta-2 microglobulin amyloidosis, hereditary non-neuropathic amyloidosis and Finnish hereditary systemic amyloidosis.

11. The method of claim 1 or 2, wherein the subject has a disease or condition characterized by ocular protein aggregation.

12. The method of claim 11, wherein the disease or condition is selected from cataracts and presbyopia.

13. A method of treating a condition associated with mitochondrial dysfunction in a subject in need thereof, said method comprising administering to the subject an effective amount of a compound of a compound of formula (I), 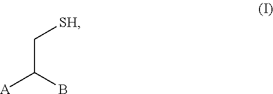
or a pharmaceutically acceptable acid addition salt thereof, in an amount sufficient to inhibit the aggregation of the protein or peptide, and wherein
(i) A is —CH₂NHR′ and B is —CH₂NHR, or A=—NRR′ and B═H; and
(ii) each of R and R′ are, independently, selected from H, C₁-C₆ alkyl, and C₁-C₆ heteroalkyl,
with the proviso that R and R′ are not both H if B═H.

14. The method of claim 13, wherein the compound is 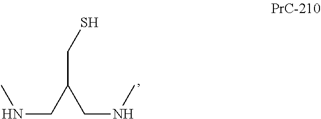
or a pharmaceutically acceptable salt thereof.

15. The method of claim 13, wherein said condition is a neurodegenerative disorder, a neuropsychiatric disorder, diabetes, metabolic disease, an ocular disorder associated with mitochondrial dysfunction, an ischemia related condition, aging, mitochondrial toxicity associated with therapeutic agents, or migraine.

16. The method of claim 15, wherein said condition is a neurodegenerative disorder selected from Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, Parkinson's disease, Alzheimer's disease, and Huntington's disease.

17. The method of claim 15, wherein said condition is a neuropsychiatric disorder selected from bipolar disorder, schizophrenia, depression, addiction disorders, anxiety disorders, attention deficit disorders, personality disorders, autism, and Asperger's Syndrome.

18. The method of claim 15, wherein said condition is an ocular disorder associated with mitochondrial dysfunction selected from glaucoma, diabetic retinopathy and age-related macular degeneration.

19. The method of claim 15, wherein said condition is an ischemia related condition resulting from vascular occlusion, arteriosclerosis, heart valve diseases, tachycardia, hypertension and hypotension.

20. A method of treating a chronic inflammatory condition in a subject in need thereof, said method comprising administering to the subject an effective amount of a compound of a compound of formula (I), 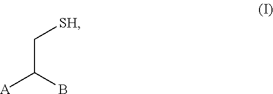
or a pharmaceutically acceptable acid addition salt thereof, in an amount sufficient to inhibit the aggregation of the protein or peptide, and wherein
(i) A is —CH₂NHR′ and B is —CH₂NHR, or A=—NRR′ and B═H; and
(ii) each of R and R′ are, independently, selected from H, C₁-C₆ alkyl, and C₁-C₆ heteroalkyl,
with the proviso that R and R′ are not both H if B═H.

21. The method of claim 20, wherein the compound is 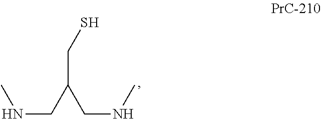
or a pharmaceutically acceptable salt thereof.

22. The method of claim 20, wherein said chronic inflammatory condition is selected from rhinosinusitis, arthritis, dermatitis, vasculitis, acute malaria, sickle cell disease, a gastrointestinal inflammatory condition, or an inflammatory pulmonary condition.

23. The method of claim 22, wherein said chronic inflammatory condition is an inflammatory pulmonary condition selected from asthma, chronic obstructive pulmonary disease (COPD), physical trauma-induced pulmonary conditions, emphysema, bronchitis, sarcoidosis, histiocytosis, lymphangiomyomatosis, acute lung injury, chronic lung disease, bronchopulmonary dysplasia, pneumonia, airway exacerbations, and acute respiratory distress syndrome (ARDS).

24. The method of claim 22 wherein said chronic inflammatory condition is a gastrointestinal inflammatory condition selected from inflammatory bowel disease (IBD), ulcerative colitis (UC), Crohn's disease (CD), colitis, and chronic hepatis, such as viral hepatitis, alcohol related hepatitis and non-alcoholic fatty hepatitis.

25. The method of claim 20, wherein said chronic inflammatory condition is an autoimmune disease.

26. The method of claim 25, wherein said an autoimmune disease is selected from multiple sclerosis, diabetes Type I, rheumatoid arthritis, juvenile rheumatoid arthritis, psoriatic arthritis, pancreatitis, psoriasis, plaque psoriasis, guttate psoriasis, inverse psoriasis, pustular psoriasis, erythrodermic psoriasis, paraneoplastic autoimmune diseases, autoimmune hepatitis, bullous pemphigoid, myasthenia gravis, inflammatory bowel disease, Crohn's disease, ulcerative colitis, celiac disease, thyroiditis, Sjogren's syndrome, Guillain-Barre disease, Raynaud's phenomenon, Addison's disease, primary biliary cirrhosis, primary sclerosing cholangitis, non-alcoholic fatty liver disease, and non-alcoholic steatohepatitis, and diabetes.

27. The method of claim 20, wherein said chronic inflammatory condition is a neuroinflammatory disease.

28. The method of claim 27, wherein said neuroinflammatory disease is selected from Multiple sclerosis, diabetes Type I, neuromyelitis optica, anti-myelin oligodendrocyte glycoprotein antibody disorder, autoimmune encephalitis, transverse myelitis, optic neuritis and neurosarcoidosis or is a neurodegenerative disorder selected from Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, Parkinson's disease, Alzheimer's disease, and Huntington's disease.
